(19) United States
(12) Patent Application Publication
(10) Pub. No.: US 2020/0155607 A1
(43) Pub. Date: May 21, 2020

(54) Enriched Cellular Compositions and Therapeutic Use

(71) Applicant: Abraham J and Phyllis Katz Cord Blood Foundation, Cleveland, OH (US)

(72) Inventors: Mary Laughlin, Cleveland, OH (US); Jennifer Greene-Roos, Cleveland, OH (US)

(21) Appl. No.: 16/608,285

(22) PCT Filed: Apr. 25, 2018
(86) PCT No.: PCT/US2018/029256

§ 371 Date: Oct. 25, 2019

Related U.S. Application Data
(60) Provisional application No. 62/489,574, filed on Apr. 25, 2017.
(60) Provisional application No. 62/550,919, filed on Aug. 28, 2017.
(60) Provisional application No. 62/610,624, filed on Dec. 27, 2017.

(51) Int. Cl. A61K 35/19; C12N 5/077; A61P 17/02; A61K 35/15; A61K 35/16

(52) U.S. Cl. CPC A61K 35/19; C12N 5/0655; A61K 35/16; A61K 35/15; A61P 17/02

(57) ABSTRACT

Methods for providing wound healing compositions that include umbilical cord blood derived monocytes and umbilical cord blood derived platelet rich plasma are provided. Methods for treating a non-healing wound in a subject by administration of a wound healing composition that includes umbilical cord blood derived monocytes and umbilical cord blood derived platelet rich plasma are provided.

### CROSS REFERENCE TO RELATED APPLICATIONS

This application claims the priority benefit of U.S. Provisional Patent Application No. 62/489,574 filed on Apr. 25, 2017, U.S. Provisional Patent Application No. 62/550,919 filed on Aug. 28, 2017, and U.S. Provisional Patent Application No. 62/610,624 filed on Dec. 27, 2017, the entire contents of which is incorporated herein by reference.

### FIELD OF THE INVENTION

The present disclosure relates generally to wound healing compositions and methods of providing the same. The present disclosure also relates to methods for treating a non-healing wound in a patient by administration of a topical wound healing composition.

### BACKGROUND

Wound healing is a dynamic process leading to restoration of tissue integrity and function. The wound healing process consists of four highly integrated and overlapping phases: hemostasis, inflammation, proliferation, and tissue remodeling or resolution. These phases and their biophysiological functions ordinarily occur in a specific sequence, at specific times, and continue for a specific duration at an optimal intensity.

Chronic wounds are wounds that exhibit impaired healing as a result of failing to progress through the normal stages of healing. Chronic wounds frequently enter a state of pathologic inflammation due to a postponed, incomplete, or uncoordinated healing process. Most chronic wounds can be classified into three categories: venous/arterial ulcers, diabetic ulcers, and pressure ulcers.

Many factors can contribute to poor wound healing. Local factors include wound infection, ischemia, tissue necrosis, foreign bodies, and edema. Systemic factors can include inflammation, diabetes, malnutrition, metabolic diseases, immunosuppression, smoking, age, and alcohol. Wound infection is a particularly common reason for poor wound healing. While all wounds are contaminated with bacteria, whether a wound becomes infected is ultimately determined by the hoses immune competence, the type of wound-pathogen(s) present, the formation of a microbial biofilm, and/or the numbers of bacteria present.

Chronic wounds represent a significant burden both financially and in terms of lost quality of life, and current wound management strategies do not adequately treat chronic wounds. For example, 15-27% of diabetic patients with chronic ulcers require limb amputation despite standard clinical treatment including wound dressing, debridement of necrotic tissue, and offloading. Infection accounts for 50% of lower limb amputation. Of the patients requiring limb amputation, up to 50% require a second amputation within five years of the first. The five year survival rate for patients receiving an amputation is 27%. Additional examples of chronic non-healing wounds includes patients with peripheral vascular disease and patients with Sickle Cell Disease who experience complications with chronic non-healing leg ulcers occurring in 10-65% of patients.

Therefore, there is a need for more effective compositions and approaches to treating chronic wounds that control or eliminate wound bioburden while promoting normal tissue regeneration.

### SUMMARY OF THE INVENTION

The disclosure provides methods for producing a wound healing composition. The methods can include one or more of the following steps: producing platelet rich plasma from umbilical cord blood; isolating monocytes from umbilical cord blood; stimulating the isolated monocytes with a monocyte adjuvant; culturing the isolated monocytes short-term in media with cytokines added; cryopreserving the platelet rich plasma and the isolated monocytes; thawing the platelet rich plasma and the monocytes; and combining the platelet rich plasma and the isolated monocytes.

In some embodiments, the monocyte adjuvant induces a hypoxic response in the monocytes. In some embodiments, the monocyte adjuvant is a hypoxia inducible factor (HIF) modulator. In some embodiments, the monocyte adjuvant is a toll-like receptor 4 (TLR4) modulator. In some embodiments, the monocyte adjuvant is selected from deferasirox, deferiprone, deferoxamine, cobalt chloride, and monophosphoryl lipid A. In some embodiments, thrombin is added to the combined platelet rich plasma and isolated monocytes to form a gel. In some embodiments, the isolated monocytes are stimulated in the presence of the platelet rich plasma. In some embodiments, the isolated monocytes are cultured short term (3-10 days) in media containing additives including cytokines such as M-CSF.

In embodiments, the invention provides a method for producing a wound healing composition by producing platelet rich plasma from umbilical cord blood, isolating monocytes from umbilical cord blood, and stimulating the isolated monocytes.

In other embodiments, the invention provides a method for producing a wound healing composition by producing platelet rich plasma from umbilical cord blood, isolating monocytes from umbilical cord blood, and stimulating the isolated monocytes, wherein the isolated monocytes are stimulated with a short term culture in vitro.

In other embodiments, the invention provides a method for producing a wound healing composition by producing platelet rich plasma from umbilical cord blood, isolating monocytes from umbilical cord blood, and stimulating the isolated monocytes, wherein the isolated monocytes are stimulated with a monocyte adjuvant.

In embodiments, the invention provides a method for producing a wound healing composition by producing platelet rich plasma from umbilical cord blood, isolating monocytes from umbilical cord blood, and stimulating the isolated monocytes, wherein the isolated monocytes are stimulated with a monocyte adjuvant and with a short term culture in vitro.

In other embodiments, the invention provides a method for producing a wound healing composition by producing platelet rich plasma from umbilical cord blood, isolating monocytes from umbilical cord blood, and stimulating the isolated monocytes, wherein the isolated monocytes are stimulated with a monocyte adjuvant that induces a hypoxic response in the monocytes.

In other embodiments, the invention provides a method for producing a wound healing composition by producing platelet rich plasma from umbilical cord blood, isolating monocytes from umbilical cord blood, and stimulating the isolated monocytes, wherein the isolated monocytes are stimulated with a hypoxia inducible factor (HIF) modulator.

In other embodiments, the invention provides a method for producing a wound healing composition by producing platelet rich plasma from umbilical cord blood, isolating monocytes from umbilical cord blood, and stimulating the isolated monocytes, wherein the isolated monocytes are stimulated with a toll-like receptor 4 (TLR4) modulator.

In other embodiments, the invention provides a method for producing a wound healing composition by producing platelet rich plasma from umbilical cord blood, isolating monocytes from umbilical cord blood, and stimulating the isolated monocytes, wherein the isolated monocytes are stimulated with either deferasirox, deferiprone, deferoxamine, cobalt chloride, monophosphoryl lipid A, or a combination thereof.

In embodiments, the invention provides a method for producing a wound healing composition by producing platelet rich plasma from umbilical cord blood, isolating monocytes from umbilical cord blood, and stimulating the isolated monocytes, wherein the platelet rich plasma and the isolated monocytes are combined.

In embodiments, the invention provides a method for producing a wound healing composition by producing platelet rich plasma from umbilical cord blood, isolating monocytes from umbilical cord blood, and stimulating the isolated monocytes, wherein the platelet rich plasma and the isolated monocytes are combined and form a gel.

In embodiments, the invention provides a method for producing a wound healing composition by producing platelet rich plasma from umbilical cord blood, isolating monocytes from umbilical cord blood, stimulating the isolated monocytes, cryopreserving the platelet rich plasma and the isolated monocytes, thawing the platelet rich plasma and the monocytes, and combining the platelet rich plasma and the isolated monocytes.

In embodiments, the invention provides a method for producing a wound healing composition by producing platelet rich plasma from umbilical cord blood, isolating monocytes from umbilical cord blood, and stimulating the isolated monocytes, wherein the isolated monocytes are stimulated in the presence of the platelet rich plasma.

In embodiments, the invention provides a method for producing a wound healing composition by producing platelet rich plasma from umbilical cord blood, isolating monocytes from umbilical cord blood, stimulating the isolated monocytes, and adding mesenchymal stem cells to the wound healing composition.

In embodiments, the invention provides a therapeutic composition for wound healing comprising, platelet rich plasma derived from umbilical cord blood, and monocytes derived from umbilical cord blood, wherein the monocytes have been stimulated with a monocyte adjuvant.

In other embodiments, the invention provides a therapeutic composition for wound healing comprising, platelet rich plasma derived from umbilical cord blood, mesenchymal stem cells, and monocytes derived from umbilical cord blood, wherein the monocytes have been stimulated with a monocyte adjuvant.

In embodiments, the invention provides a method for treating a non-healing wound in a subject, comprising administering to the subject a composition comprising a therapeutically effective dose of umbilical cord blood derived platelet rich plasma and umbilical cord blood derived monocytes stimulated with a monocyte adjuvant and/or short term culture in vitro.

In embodiments, the invention provides a method for treating a chronic wound in a subject, comprising administering to the subject a composition comprising a therapeutically effective dose of umbilical cord blood derived platelet rich plasma and umbilical cord blood derived monocytes stimulated with a monocyte adjuvant and/or short term culture in vitro.

In embodiments, the invention provides a method for treating a diabetic ulcer, a decubitus ulcer, a venous ulcer, an arterial ulcer, an infectious ulcer, a burn ulcer, a trauma-induced ulcer, or a surgical wound in a subject, comprising administering to the subject a composition comprising a therapeutically effective dose of umbilical cord blood derived platelet rich plasma and umbilical cord blood derived monocytes stimulated with a monocyte adjuvant and/or short term culture in vitro.

In embodiments, the invention provides a method for treating a chronic wound in a subject, comprising administering to the subject a composition comprising a therapeutically effective dose of umbilical cord blood derived platelet rich plasma and umbilical cord blood derived monocytes stimulated with a monocyte adjuvant and/or short term culture in vitro, wherein the platelet rich plasma and the monocytes are derived from autologous or allogeneic umbilical cord blood.

In embodiments, the invention provides a method for treating a chronic wound in a subject, comprising administering to the subject a composition comprising a therapeutically effective dose of umbilical cord blood derived platelet rich plasma and umbilical cord blood derived monocytes stimulated with deferoxamine, monophosphoryl lipid A, or a combination thereof, and/or short term culture in vitro.

In embodiments, the invention provides a method for treating a chronic wound in a subject, comprising administering to the subject a composition comprising a therapeutically effective dose of umbilical cord blood derived platelet rich plasma, mesenchymal stem cells, and umbilical cord blood derived monocytes stimulated with a monocyte adjuvant and/or short term culture in vitro.

The disclosure also provides therapeutic compositions for wound healing. In some embodiments, the compositions include: platelet rich plasma derived from umbilical cord blood; and monocytes derived from umbilical cord blood, wherein the monocytes have been stimulated with a monocyte adjuvant and/or cultured ex vivo short-term in media with additives including cytokines.

The disclosure also provides methods for treating a non-healing wound in a subject. In some embodiments, the methods include: administering to the subject a composition comprising a therapeutically effective dose of umbilical cord blood derived platelet rich plasma and umbilical cord blood derived monocytes stimulated with a monocyte adjuvant and/or monocytes cultured short term ex vivo. In some embodiments, the non-healing wound is a chronic wound. In some embodiments, the nonhealing wound is a diabetic ulcer, a decubitus ulcer, a venous ulcer, an arterial ulcer, an infectious ulcer, a burn ulcer, a trauma-induced ulcer, or a surgical wound. In some embodiments, the platelet rich plasma and the monocytes are derived from allogeneic umbilical cord blood. In some embodiments, the monocyte adjuvant is deferoxamine, monophosphoryl lipid A, or a combination thereof. In some embodiments, the monocytes are cultured for 3-10 days in media containing additives including M-CSF.

In embodiments, the invention provides pharmaceutical compositions, and methods for the manufacture of pharmaceutical compositions, for all of the methods of treatment disclosed herein.

### BRIEF DESCRIPTION OF THE DRAWINGS

FIGS. 1A-1B depict the purity and vitality of monocytes isolated in accordance with embodiments of this disclosure. FIG. 1A relates to monocyte purity.

FIG. 1B relates to monocyte viability.

FIG. 2 depicts the effect of umbilical platelet rich plasma (uPRP) on monocyte growth factor production.

FIGS. 3A-3B depict a BrdU proliferation assay (FIG. 3A) and a wound scratch assay (FIG. 3B).

FIGS. 4A-4B depict the effect of deferoxamine (DFO) stimulated monocytes on fibroblast function. FIG. 4A depicts the impact of DFO on fibroblast migration. FIG. 4B depicts the proliferation of human dermal fibroblasts (HDFs) in response to PRP and monocytes.

FIGS. 5A-5B depict the effect of mitomycin c on human dermal fibroblast proliferation.

FIG. 6 depicts a schematic of a bactericidal assay.

FIG. 7 depicts a bactericidal assay investigating the ability of umbilical cord blood derived monocytes to kill Pseudomonas aeruginosa (P. aeruginosa).

FIGS. 8A and 8B depict TNF-α production by UCB and AB monocytes in response to LPS and P. aeruginosa. FIG. 8A shows the response from UCB monocytes stimulated with LPS, or P. aeruginosa. FIG. 8B shows the response from AB monocytes stimulated with LPS, or P. aeruginosa.

FIGS. 9A and 9B depict nitrite production by UCB and adult monocytes in response to LPS and P. aeruginosa. FIG. 9A shows the response from UCB monocytes stimulated with LPS, or P. aeruginosa. FIG. 9B shows the response from adult monocytes stimulated with LPS, or P. aeruginosa.

FIG. 10 depicts in vitro TNF-α production by monocytes in the absence of PRP in response to P. aeruginosa.

FIG. 11 depicts in vitro nitrite production by monocytes in the absence of PRP in response to LPS and P. aeruginosa.

FIG. 12 depicts TNF-α production by monocyte derived macrophages in response to LPS and P. aeruginosa.

FIG. 13 depicts nitrite production by monocyte derived macrophages in response to LPS and P. aeruginosa.

FIG. 14 depicts VEGF production by monocyte differentiated macrophages in response to LPS and P. aeruginosa.

FIG. 15 depicts a MATRIGEL angiogenesis assay measuring the ability of HUVECs to form endothelial tubules on a MATRIGEL matrix in response to stimuli.

FIGS. 16A and 16B depict images from an in vivo preclinical assay testing CORDHEAL (UCB derived monocytes stimulated with DFO and PRP) in a murine splinted excisional biopsy model.

FIGS. 17A-17D depict data where genetically diabetic (db/db) mice with wounds on the dorsum which were splinted open to promote secondary intention.

### DETAILED DESCRIPTION

I. Overview

The present disclosure provides compositions and methods for treating non-healing wounds. The disclosure also provides methods of making these compositions. The compositions generally include monocytes and platelet rich plasma, both of which are derived from umbilical cord blood. The monocytes and/or platelet rich plasma can be stimulated and/or cultured in vitro to enhance their therapeutic properties. The compositions combine the growth factor production of platelet rich plasma with the antibacterial and wound healing properties of neonatal monocytes to target both cellular dysfunction and infection leading to ineffective wound healing.

Chronic wounds are characterized by dysfunctional granulation tissue formation, impaired angiogenesis, and reduced localized expression of growth factors. Topical application of umbilical cord derived platelet rich plasma and monocytes augments healing of chronic wounds. For example, topically applied umbilical cord derived platelet rich plasma and umbilical cord derived monocytes reduce time to complete wound healing via paracrine and direct cell-mediated bactericidal and effector mechanisms on fibroblasts and endothelial cells. This, in turn, correlates with clearance of wound site infection and necrosis, enhanced granulation tissue formation, and enhanced neovasculogenesis.

Platelets and monocytes produce significant amounts of growth factors, such as VEGF, PDGF, TGF-β1, and bFGF, which serve to enhance wound healing by mediating fibroblast and endothelial cell proliferation, migration, and angiogenesis. Growth factor production is synergistically enhanced when umbilical cord blood derived monocytes (UCB monocytes) are co-cultured in vitro with umbilical cord blood derived platelet rich plasma (UCB PRP). Further, both platelet rich plasma and monocytes have antibacterial properties which reduce and/or resolve wound site infection to allow the normal process of wound healing to proceed.

The present disclosure flows from the combination of multiple novel findings. First, the functional properties of umbilical cord blood derived monocytes and platelet rich plasma are surprisingly synergistically enhanced when used in combination with one another relative to monotherapy. Second, it has been surprisingly found that desferoxamine exerts an effect on monocytes that mimics hypoxia and enhances their biologic function with respect to wound healing, including angiogenesis. Third, it has been surprisingly found that umbilical cord blood derived monocytes improve wound healing relative to adult derived monocytes because umbilical cord blood derived monocytes have unique wound healing capabilities and do not induce fibrosis. Fourth, it has been surprisingly found that umbilical cord blood derived platelet rich plasma, relative to adult derived platelet rich plasma, has a higher concentration of cytokines and other biologically active factors that enhance monocyte viability and function. Fifth, it has been surprisingly found that short-term in vitro culture of UCB monocytes does not diminish their potency in wound healing properties.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, “umbilical cord blood” refers to a source of pluripotent and/or multipotent stem cells obtained from the blood of umbilical cords that are left over after birth. Umbilical cord blood includes blood obtained from a neonate. Umbilical cord blood also refers to blood obtained from the umbilical cord or placenta of newborns.

As used herein, “umbilical cord blood unit” refers to a volume of umbilical cord blood that is collected from a single donor.

As used herein, “umbilical cord tissue” generally refers to tissue from an umbilical cord such as umbilical vein sub-endothelium, umbilical cord blood, amnion, placenta, amniotic fluid, microvillus, and Wharton's jelly.

As used herein, “platelet rich plasma” or “PRP” refers to a volume of plasma that has a platelet concentration above baseline. Normal platelet counts in blood range between 150,000/microliter and 350,000/microliter. Platelet rich plasma typically has an increased platelet concentration of about a 1.5-20 fold increase as compared to venous blood. The platelet concentration is specifically increased by any suitable method (e.g. centrifugation, fractionation, separation). For example, platelet enriched plasma can be obtained by double centrifugation designed to separate a PRP aliquot from platelet-poor plasma and red blood cells. Platelet rich plasma may or may not include white blood cells.

As used herein, a composition containing a “purified cell population” or “purified cell composition” means that at least 30%, 50%, 60%, typically at least 70%, and more preferably 80%, 90%, 95%, 98%, 99%, or more of the cells in the composition are of the identified type.

As used herein, “substantially separated from” or “substantially separating” refers to the characteristic of a population of first substances being removed from the proximity of a population of second substances, wherein the population of first substances is not necessarily devoid of the second substance, and the population of second substances is not necessarily devoid of the first substance. However, a population of first substances that is “substantially separated from” a population of second substances has a measurably lower content of second substances as compared to the non-separated mixture of first and second substances. In one aspect, at least 30%, 50%, 60%, 70%, 80%, 90%, 95%, 98%, 99%, or more of the second substance is removed from the first substance.

As used herein, “patient” or “subject” means an animal subject to be treated, with human patients being preferred.

As used herein, the term “wound” includes an injury to the skin and/or subcutaneous tissue. Wounds can be superficial (loss of epidermis only), partial thickness (involves the epidermis and dermis), or full thickness (involves the dermis, subcutaneous tissue, and sometimes bone). Examples of wounds can include burns, incisions, excisions, lacerations, abrasions, surgical wounds, and ulcers.

As used herein, the term “non-healing wound” refers to a wound that does not heal at a typical rate. Examples of non-healing wounds include delayed-healing wounds, incompletely healing wounds, and chronic infected wounds. Non-healing wounds can be characterized as having: (1) a prolonged inflammatory phase, (2) a slow forming extracellular matrix, and/or (3) a decreased rate of epithelialization or closure.

As used herein, the term “chronic wound” refers to a wound that has not healed within three months, or likely will not heal within three months. A chronic wound can be characterized as having: (1) a chronic self-perpetuating state of wound inflammation; (2) a deficient and defective wound extracellular matrix; (3) poorly responding (senescent) wound cells (including fibroblasts); (4) limited extracellular matrix production; and/or (5) failure of reepithelialization due in part to lack of the necessary extracellular matrix orchestration and lack of scaffold for migration. Chronic wounds can also be characterized as having (1) prolonged inflammation and proteolytic activity leading to ulcerative lesions; (2) progressive deposition of matrix in the affected area, (3) longer repair times, (4) less wound contraction, (5) slower reepithelialization, and (6) increased thickness of granulation tissue.

As used herein, “inducing a skin wound healing process” refers to the induction of granulation tissue formation for wound contraction or the induction of epithelialization. Wound healing can be conveniently measured by decreasing wound area.

As used herein, “accelerating a skin wound healing process” refers to the acceleration of granulation tissue formation for wound contraction or the acceleration of epithelialization. Wound healing can be conveniently measured by decreasing wound area.

As used herein, “a monocyte adjuvant” refers to a molecule that induces a hypoxic response in a monocyte. Examples of monocyte adjuvants include molecules that increase the expression and/or activity of hypoxia inducible factor (e.g. HIF-1α) and molecules that increase the expression and/or activity of Toll-like receptor 4 (TLR4).

As used herein, “therapeutically effective” refers to an amount of a substance (cells, biomolecules, etc.) that is sufficient to treat or ameliorate, or in some manner reduce the symptoms associated with a disease or condition. When used with reference to a method, the method is sufficiently effective to treat or ameliorate, or in some manner reduce the symptoms associated with a disease or condition. For example, an effective amount in reference to a disease is that amount which is sufficient to block or prevent its onset; or if disease pathology has begun, to palliate, ameliorate, stabilize, reverse or slow progression of the disease, or otherwise reduce pathological consequences of the disease. In any case, an effective amount may be given in single or divided doses.

As used herein, the term “treatment” embraces at least an amelioration of the symptoms associated with a disease or condition in the patient, where amelioration is used in a broad sense to refer to at least a reduction in the magnitude of a parameter, e.g. a symptom associated with the condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

Throughout this disclosure the methods and compositions are described in reference to umbilical cord blood. It will be appreciated by those of ordinary skill that umbilical cord tissue can, in some instances, be substituted for umbilical cord blood in the present methods and compositions. Thus, the methods and compositions of the present disclosure equally apply to umbilical cord tissue as they do to umbilical cord blood.

II. Methods for Producing Wound Healing Compositions

Methods are provided for producing a wound healing composition. In embodiments, the methods include one or more of the following steps: producing platelet rich plasma from umbilical cord blood; separating/isolating/purifying monocytes from umbilical cord blood; conditioning the monocytes ex vivo (e.g. stimulating the monocytes with a monocyte adjuvant); storing the platelet rich plasma and the monocytes (e.g. cryopreserving); and/or combining the platelet rich plasma and the monocytes to form a single composition.

In some embodiments, platelet rich plasma is produced from umbilical cord blood. Methods for producing platelet rich plasma from umbilical cord blood are well known in the art (e.g. differential centrifugation). For example, platelet rich plasma can be produced by separating red blood cells with an initial centrifugation, concentrating platelets with a second centrifugation, and then suspending the platelets in a small final plasma volume.

In some embodiments, monocytes are separated, isolated, and/or purified from umbilical cord blood. In some embodiments, monocytes are substantially separated from other cells in umbilical cord blood to form a purified monocyte population. Methods for separating/isolating/purifying monocytes from blood are well known in the art. One exemplary technique can include Ficoll-Paque density gradient separation to isolate viable mononuclear cells from blood using a centrifugation procedure, and affinity separation to separate monocytes from the mononuclear cells. Exemplary affinity separation techniques can include, for example, magnetic separation (e.g. antibody-coated magnetic beads) and fluorescence-activated cell sorting.

In one non-limiting example, mononuclear cells can be obtained from umbilical cord blood by gradient density separation using Ficoll. Monocytes can then be isolated by depletion of non-monocytes (negative selection) from the mononuclear cells. Non-monocytes can be indirectly magnetically labeled with a cocktail of biotin-conjugated monoclonal antibodies, as a primary labeling reagent, and anti-biotin monoclonal antibodies conjugated to microbeads, as a secondary labeling reagent. The magnetically labeled non-monocytes can be depleted by retaining them on a MACS (magnetically assisted cell sorting) column in the magnetic field of a MACS separator, while the unlabeled monocytes pass through the column. This process can leave behind an enriched/purified population of monocytes. In some embodiments the monocytes are isolated by adherence to the column. In other, monocytes can be separated from other cells in a mononuclear preparation by use of a flow cytometric cell sorter. In some embodiments, at least 75%, 85%, 86%, 87%, 88%, 89%, 90%, 91%, 92%, 93%, 94%, 95%, 96%, 97%, 98%, 99%, or more of the cells of the resulting composition are monocytes. In some embodiments, the purity of monocytes is equal to or greater than 75%, 85%, 86%, 87%, 88%, 89%, 90%, 91%, 92%, 93%, 94%, 95%, 96%, 97%, 98%, 99%, or more.

In some embodiments, monocytes are conditioned ex vivo. In some embodiments, ex vivo conditioning of monocytes is one of the ways to prepare monocytes for transplantation and/or treatment. Suitable methods for conditioning monocytes are known to those of ordinary skill. In some embodiments, monocytes are conditioned by stimulating the monocytes with a monocyte adjuvant. Monocyte adjuvants can include molecules that induce a hypoxic response in a monocyte. Examples of monocyte adjuvants can include hypoxia inducible factor (HIF) modulators and toll-like receptor 4 (TLR4) modulators. Examples of monocyte adjuvants can include molecules that increase the expression and/or activity of hypoxia inducible factor (e.g. HIF-1α) and molecules that increase the expression and/or activity of Toll-like receptor 4 (TLR4). Examples of monocyte adjuvants can include deferasirox, deferiprone, deferoxamine, cobalt chloride, and monophosphoryl lipid A. In some embodiments, monocytes are conditioned in the presence of platelet rich plasma (e.g. monocytes are cultured with platelet rich plasma). In some embodiments, monocytes are cultured ex vivo for 3-10 days in media containing additives that enhance monocyte wound healing, angiogenesis, phagocytosis, and bactericidal function.

In some embodiments, compositions comprising monocytes contain a clinically relevant number or population of monocytes. In some embodiments, the compositions include about 10³, about 10⁴, about 10⁵ cells, about 10⁶ cells, about 10⁷ cells, about 10⁸ cells, about 10⁹ cells, about 10¹⁰ cells or more. In some embodiments, the number of monocytes present in the composition will depend upon the ultimate use for which the composition is intended, e.g., the disease or state or condition, patient condition (e.g., size, weight, health, etc.), and other health related parameters that a skilled artisan would readily understand. In addition, in some embodiments, the clinically relevant number of cells can be apportioned into multiple doses that cumulatively equal or exceed the desired administration, e.g., 10⁹ or 10¹⁰ cells.

In some embodiments, monocytes and/or platelet rich plasma are stored for later use (e.g. refrigerated, frozen, or cryopreserved). Methods for storing monocytes and/or platelet rich plasma from blood are well known in the art. In embodiments where monocytes and/or platelet rich plasma are stored for later use, the monocytes and/or platelet rich plasma can be thawed or warmed at a later date. In some embodiments, monocytes and/or platelet rich plasma are provided for immediate use (e.g. not stored).

In some embodiments, monocytes and/or platelet rich plasma are combined to form a single composition. The monocytes and/or platelet rich plasma can be combined before, at the same time as, or after the conditioning of monocytes. In some embodiments, the monocytes and/or platelet rich plasma can be combined before being stored for later use. In some embodiments, the monocytes and/or platelet rich plasma can be combined after having been stored for a period of time. In some embodiments, the combined monocytes and/or platelet rich plasma form a gel.

In some embodiments, umbilical cord blood can originate from a variety of animal sources including, for example, humans. In some embodiments, umbilical cord blood can originate from a person to be treated (i.e. autologous umbilical cord blood). In some embodiments, umbilical cord blood can be immunocompatible with a person to be treated (i.e. allogeneic umbilical cord blood).

In some embodiments, compositions comprising monocytes and/or platelet rich plasma are provided. In some embodiments, compositions comprising substantially purified monocytes and/or substantially purified platelet rich plasma are provided. In some embodiments, the monocytes and/or platelet rich plasma are autologous or allogeneic.

The compositions comprising monocytes and/or platelet rich plasma can be provided to any suitable person or entity such as, for example, a patient, a clinician treating the patient, or a biological bank.

In some embodiments, compositions comprising monocytes and/or platelet rich plasma are non-naturally occurring. In some embodiments, compositions comprising monocytes and/or platelet rich plasma are not naturally occurring because the monocytes and/or platelet rich plasma are the result of one or more of purification, ex vivo conditioning, and the like.

In some embodiments, a therapeutic composition comprising a therapeutically effective dose of monocytes and/or platelet rich plasma is provided. In some embodiments, a therapeutic composition comprising a therapeutically effective dose of substantially purified monocytes and/or substantially purified platelet rich plasma is provided.

III. Exemplary Uses of Wound Healing Compositions

Methods of treating non-healing wounds are provided. In some embodiments, a subject is identified as requiring a wound healing composition. A subject can require a wound healing composition to treat a wound such as a non-healing wound or a chronic wound. Exemplary non-healing wounds include a diabetic ulcer, a decubitus ulcer, a venous ulcer, an arterial ulcer, an infectious ulcer, a burn ulcer, a trauma-induced ulcer, and a surgical wound. In embodiments, when a subject is identified as requiring a wound healing composition, the methods can include screening umbilical cord blood units, typically stored in umbilical cord blood banks, to identify donor umbilical cord blood units that are immunologically compatible with the subject (e.g. allogeneic or autologous).

Methods are provided for treating a non-healing wound in a subject. In some embodiments, the methods include administering to the subject a composition comprising a therapeutically effective dose of umbilical cord blood derived platelet rich plasma and umbilical cord blood derived monocytes stimulated with a monocyte adjuvant and/or cultured short-term in vitro. In some embodiments, the non-healing wound is a chronic wound. In some embodiments, the non-healing wound is a diabetic ulcer, a decubitus ulcer, a venous ulcer, an arterial ulcer, an infectious ulcer, a burn ulcer, a trauma-induced ulcer, or a surgical wound.

In some embodiments, a therapeutically effective amount of umbilical cord blood derived platelet rich plasma and umbilical cord blood derived monocytes stimulated with a monocyte adjuvant and/or cultured in vitro can be administered to a subject with a pharmaceutically acceptable carrier or additional UCB derived cells such as mesenchymal stromal cells. Administration routes may include any suitable means, including, but not limited to, topical application to a wound, or injection into a wound. In some embodiments, the particular mode of administration selected will depend upon the particular treatment, disease state or condition of the patient, the nature or administration route of other drugs or therapeutics administered to the subject.

In some embodiments, umbilical cord blood derived platelet rich plasma and umbilical cord blood derived monocytes stimulated with a monocyte adjuvant can be administered to a subject in a single dose or in several doses over selected time intervals, e.g., to titrate the dose. In some embodiments, administration of umbilical cord blood derived platelet rich plasma and umbilical cord blood derived monocytes stimulated with a monocyte adjuvant and/or cultured in vitro induces a skin wound healing process. In some embodiments, administration of umbilical cord blood derived platelet rich plasma and umbilical cord blood derived monocytes stimulated with a monocyte adjuvant and/or cultured in vitro accelerates a skin wound healing process.

In embodiments, the stimulated monocytes and compositions enhance anastomosis through direct cellular contact with endothelial cells, thereby promoting angiogenesis within the wound site. In embodiments, the stimulated monocytes and compositions have inherent antimicrobial functions. In embodiments, the stimulated monocytes and compositions express synergistically high levels of growth factors necessary for wound healing.

In embodiments, the invention can be provided in two frozen components, which are thawed, combined and applied to the wound. In embodiments, the combined monocytes and platelet rich plasma product gels or solidifies within 1-5 minutes, or 1-2 minutes of application in vivo within the wound bed.

In embodiments, the compositions of the invention can treat a wide spectrum of wounds at various stages of healing, including DFUs, VLUs, arterial ulcers, dehisced surgical wounds, traumatic injury wounds, burn wounds, and pressure sores. The compositions of the invention can also be used to treat patients with tunneling, sinus tracts, and bone and tendon exposure. This product can be used on patients with biofilm infected wounds.

In embodiments, a mean ulcer size in humans is between about 1.9 cm²−41.5 cm². Therefore, at a monocyte frequency of about 1×10⁶/cm², a dosage of 2×10⁶−4.1×10⁷ monocyte/wound can be used. Platelet yield is about 1-2×10¹⁰, which would generate a 10-20 ml size product at 1×10⁶ platelets/μl. In embodiments, based on angiogenesis studies, efficacy occurs at about 10-100 monocytes/μl.

In some embodiments, the methods for treating a non-healing wound in a subject can include standard wound care treatment steps. Standard wound care treatment steps are well known in the art. Examples of standard wound care treatment include wound cleaning (e.g. removing visible debris and necrotic tissue, removing dressing residue, removing excessive or dry crusting exudates), dressing the wound, administering antibiotic, and the like.

Other objects, advantages and features of the present invention will become apparent from the following specification taken in conjunction with the accompanying figures.

### Examples

CORDHEAL Formation Protocol

### PRP Production

PRP was produced, while keeping samples on ice as much as possible to reduce platelet activation prior to use. Initially, blood was transferred to a 50 ml conical tube and spun at 300×g (1250 rpm) for 15 min at 4° C. without brake. Followed by, a transfer of the plasma to a new tube, wherein a complete blood count (CBC) on total plasma was performed. The plasma was spun at 1600×g (2800 rpm) for 10 min forming a pellet containing platelets, and a supernatant being platelet-poor plasma (PPP). PPP was removed and stored, and the pellet was gently broken up and resuspended at 1×10⁶ platelets/μl in PPP to form platelet rich plasma (PRP). The PRP and PPP was stored overnight at 4° C.

Ficoll Separation with SEPMATE Tubes

15 ml Ficoll was added to a SEPMATE 50 tube (STEMCELL Technologies SEPMATE 50 #15450). Gradient media was carefully pipetted through the central hole of the SEPMATE tube. 30 ml of a blood sample diluted 1:1 with phosphate-buffered saline (PBS) was added to the SEPMATE tube by pipetting along the side of the tube. The SEPMATE tube was then centrifuged at 1200×g for 25 minutes at room temperature (RT) with the brake on. The generated supernatant, containing mono nuclear cells (MNCs), was removed by pipette and transferred to a new tube. After the addition of 40 ml PBS, the tube was spun at 1000 rpm/10 min at RT with brake on. The supernatant was decanted, and 5 ml lysis buffer was added to the tube which was then allowed to incubate at RT for 10 min before adding 50 ml PBS. The tube was spun at 1000 rpm/10 min at RT with brake on. The supernatant was decanted and the MNC was resuspended in 30 ml PBS.

### Monocyte Isolation by Negative Selection Using LD Columns

Cells were counted and spun at 2000 rpm/5 min forming a cell pellet. The cell pellet was resuspended in 30 μl of magnetic-activated cell sorting (MACS) buffer (PBS/0.5% FBS/2 mM EDTA) per 10⁷ total cells. 10 μl of FcR blocking reagent per 10⁷ total cells and 10 μl of Biotin-Antibody cocktail per 10⁷ total cells was added, mixed well and allowed to incubate for 10 min at 4° C. Followed by the addition of 30 μl of buffer per 10⁷ total cells, and 20 μl of anti-biotin microbeads per 10⁷ total cells. The resulting cell suspension was mixed well and allowed to incubate for an additional 15 minutes at 4-8° C. 1-2 ml of buffer was added and the cells were spun at 2000 rpm/5 min. The cell pellet was resuspended with up to 1.25 10⁸ cells per 500 μl buffer, with the buffer volume being scalable for larger numbers of cells.

### AUTOMACS Separation

A pre-separation filter (Miltenyi #130-041) was prepared and placed on an LD column. 500 μl of MACS buffer was applied right before adding cells. The LD column was prepared by adding and allowing 2 ml of MACS buffer to run through column and be discarded. A MNC suspension was added to the pre-separation filter and allowed to run through the filter and LD column into 50 ml conical tube. The pre-separation filter and LD column was then washed with 1 ml of MACS buffer by allowing it to run through column. After the removal of the pre-separation filter 1 ml of MACS buffer was added to the LD column and effluent collected in a 50 ml tube. The enriched/purified population of monocytes was added to 10 ml of PBS and counted. After an addition of 40 ml PBS the suspension was spun at 2000 rpm/5 min. The PBS was decanted and the pellet was resuspended in PBS/10% autologous PPP and stored overnight at 4° C.

### Monocyte Enrichment by Flow Sorting

Following Ficoll separation monocytes were resuspended in PBS/1% BSA. In some embodiments monocytes will be sorted based on inherent characteristics such as size and granularity. Monocytes will be run through a sorting flow cytometer and enriched. In other embodiments monocytes will be enriched based on flow cytometry sorting for cells labeled for CD14. For enriching labeled monocytes, cells will first be blocked with human AB serum for 15 min at room temperature. After enrichment cells the cells will be spun down and pelleted, and the supernatant discarded. The pelleted cells will be resuspended in PBS/1% BSA. Fluorescently labeled anti-human CD14 antibody will be added and incubated at 4° C. in the dark for 30 min. The cells will be washed with PBS, resuspended in PBS/1% BSA, and then run through a flow cytometer to enrich for monocytes.

### Monocyte Enrichment by Adherence and Short Term Culture

In yet other embodiments monocytes will be plated out in serum free Dulbecco's Modified Eagle Medium (DMEM) for 30 minutes at 37° C. at a concentration of 5×10⁵ cells/ml. Any non-adherent cells will be washed away with PBS. The media will then be replaced with RPMI/10% human AB serum, 500 μM and 10-100 ng/ml macrophage colony-stimulating factor (M-CSF). After 24 hrs the media will be replaced with RPMI/10% human AB serum and 10-100 ng/ml M-CSF. Subsequently, the media will be changed every 2-3 days for up to 10 days.

### CORDHEAL Formation

Using a 96-well plate monocytes were plated out in PRP at a concentration of 10-100 monocytes/μl, wherein 100 μl is added to each well. After the addition of DFO (500 μM) the wells were allowed to incubate at 37° C. for 5 hrs, at which point thrombin (1μ/ml) was added. After the addition of thrombin the wells were allowed to incubate overnight at 37° C. After incubation the liquid form was added to a MATRIGEL assay, proliferation assay, or wound scratch assay.

Assay Protocols

### Bactericidal Assay Protocol

8×10⁶ mononuclear cells were added per well in a 12 well plate and allowed to incubate 1 hr at 37° C. in serum free DMEM to allow adherence. The DMEM media was removed and replaced with 1.5 ml RPMI/20% autologous PPP and stimulation conditions and left overnight. The following morning the media was replaced with RPMI/10% PPP, and 3×10⁶ CFU bacteria was added to each well. The plate was spun down at 250× g for 10 min at RT to increase interaction between bacteria and cells. The spun down plates were allowed to incubate at 37° C. for 40 min to allow for phagocytosis. A 100 μl aliquot of media was reserved, as a positive control, to test for the presence of extracellular bacteria after phagocytosis. The samples were diluted 1:50 in LB, and 100 μl was added to a trypticase soy agar (TSA) plate. Colonies were counted after 24 hrs. After examining the colony forming units (CFUs) from these wells, there should be less bacteria in the wells with monocytes and bacteria as compared to the wells with bacteria only as long as phagocytosis is occurring. The wells were washed three times with PBS, while being careful not to detach monocytes. 500 μl of 1% saponin was added and the wells were allowed to incubate at 37° C. for 15 min. The cells were scraped out of each well using a cell scraper and vigorously vortexed. The lysis reaction was stopped by adding saponin/cells/bacteria mix to TSB at the 1:1 dilutions. At this time point (considered t=0), 100 μl of diluted cells/bacteria was added to an agar plate.

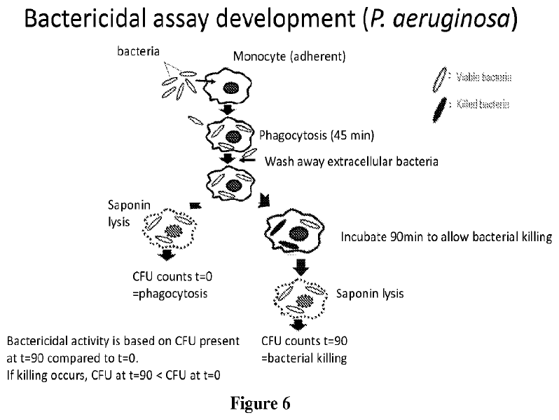

RPMI was added to the remaining infected macrophages and allowed to incubate at 37° C. for 90 min. The incubated macrophages were washed once with PBS and 500 μl of 1% saponin was added and allowed to incubate at 37° C. for 15 min. The cells were scraped out of each well using a cell scraper, and the wells were checked for cell lysis/detachment. The lysis reaction was stopped by adding saponin/cells/bacteria mix to TSB at a 1:1 dilution. At this time point (considered t=90 min), 100 μl of diluted cells/bacteria was added to an agar plate. (See FIG. 6.)

### BrdU Staining Protocol (HDFs)

10 μM BrdU was added to each sample from a stock solution of 32.5 Mm. A working stock of 1 mM in sterile PBS was made and 10 μl was added to each well, and allowed to incubate cells at 37° C. for 2 hrs. The cells were trypsinized, resuspended in FACS tubes, washed once with 2 ml FACS buffer (PBS/1% FBS), and spun down at 2000 rpm for 5 min. The cells were resuspended in 100 μl of FACS buffer.

1 ml of BrdU staining buffer was added. The BrdU concentrate should be diluted 1:4 in fix/per diluent (2 ml concentrate, 6 ml diluent). The diluted BrdU concentrate and the resuspended cells were mixed without vortexing and allowed to incubate at RT for 15 min. The cells were washed twice with flow cytometry buffer.

100 μl of DNase I working solution thawed on ice was added and allowed to incubate for 1 hr at 37° C. in the dark. The cells were washed twice with flow cytometry staining buffer, and 5 μl/sample of Anti-BrdU fluorochrome conjugated antibody was added, and allowed to incubate for 30 min at RT in the dark. The cells where then washed twice with FACS buffer and the data was collected using a LSRII flow cytometer within 24 hrs.

### MATRIGEL Assay Protocol

Phenol red free growth factor reduced MATRIGEL (Corning #356231) was thawed on ice in a fridge overnight. The MATRIGEL was diluted to 5 mg/ml in PBS and 12 μl of MATRIGEL was added to each well of u-angiogenesis slide (IBIDI #81506) while being kept on ice. After placing the lid on the slide it was placed in a petri dish with wet paper towel for added humidity. The MATRIGEL was allowed to solidify for 1 hr at 37° C., and 10,000 HUVEC in 50 μl of serum free vascular basal medium was added to the wells. Liquid from PRP and monocytes at a final concentration of 2%/well (1 μl/well) was added in accordance with plate setup, and pictures were taken every 3 hrs for 24 hrs.

### Monocyte Viability and Purity Protocol

Cells were resuspended in FACS buffer (PBS/1% FBS) at a concentration of 2×10⁵ cells/100μl, and spun down at 2000 rpm for 5 min. After removing the supernatant, the pellets were reconstituted in 100 μl FACS buffer/20% autologous PPP, vortexed, and allowed to incubate at RT for 15 min before being spun down at 2000 rpm for 5 min. The supernatant was removed and the cells stained in 100 μl of FACS buffer with the appropriate concentration of antibody (APC CD14 (Miltenyi 130-091-243)—1 μl/test, Fitc CD41a (BD #561851, clone ITGA2B)—5 μl/test), and allowed to incubate at 4° C. in the dark for 30 min. After addition of 100 μl FACS buffer, the suspension was spun down at 2000 rpm for 5 min After removing the supernatant the pellets were resuspended in 100 μl of 1× binding buffer (BD FITC Annexin V apoptosis detection kit #556547). After adding 5 μl Annexin V per well (2×10⁵ cells/test) and 5 μl PI per well (2×10⁵ cells/test) the cells were gently vortexed and allowed to incubate for 15 min at RT in the dark. 400 μl of 1× binding buffer was added to each tube and the cells were analyze by flow cytometry within 1 hr.

### Wound Scratch Assay Protocol

HDFs at 1×10⁴ cells/well were plated out in complete growth media with the culture volume being 70 μl/well in each side of culture insert (IBIDI #81176). After 24 hrs starvation media (DMEM/0.2% FBS) was added. The culture insert was then removed after 48 hrs and 2 ml PBS was added outside of well. The culture insert was removed, then PBS was removed. 10 μg/ml mitomycin c was added and allowed to incubate for 30 min at 37° C. The mitomycin c was brought up in 1 ml of DMEM/0.2% FBS without phenol red and washed once with PBS. The stimulation conditions were added to a total culture volume of 2 ml of phenol red free, serum free DMEM, wherein PRP and monocytes equal 0.2% of total culture volume. Pictures were taken every hour for 24 hrs.

### Use of CORDHEAL for Wound Healing

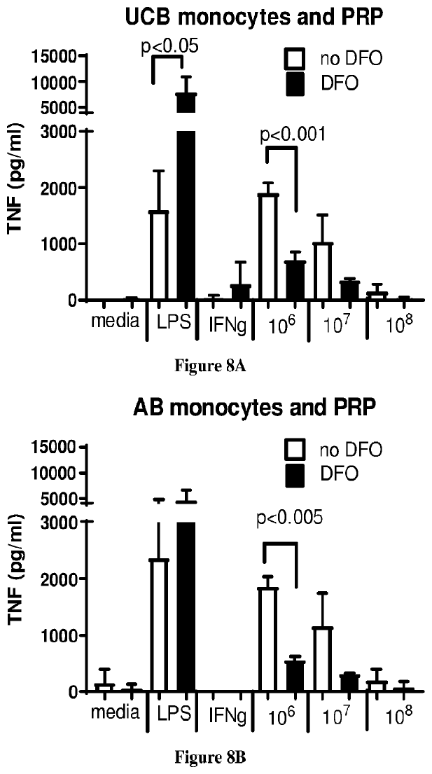

Initial studies using a murine model indicate that CORDHEAL is both safe and enhances diabetic wound healing. In vitro studies revealed that in comparison to control cells, CORDHEAL treated monocytes respond to P. aeruginosa with a diminished pro-inflammatory response as measured by TNF-α production (FIG. 8A). Interestingly, the diminished TNF-α response to bacteria seen in CORDHEAL treated monocytes is not correlated with a general immunosuppression.

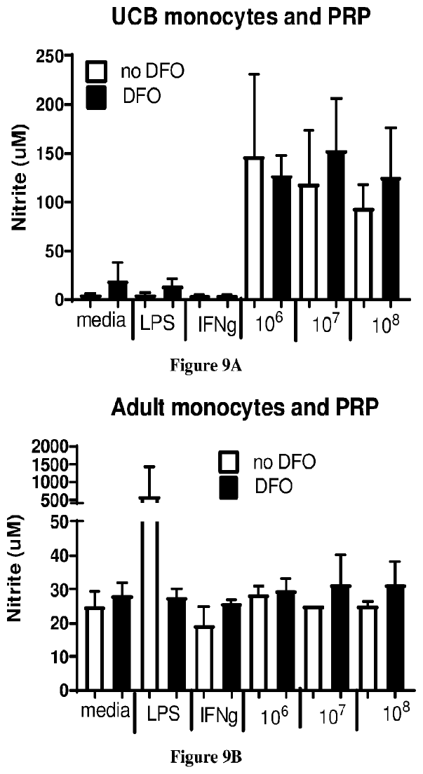

Monocytes pretreated with CORDHEAL have a strong nitric oxide (NO) response to P. aeruginosa as measured by quantitating the breakdown product nitrite (NO₂—) in a Greiss reaction (FIG. 9A). This is in contrast to the responses observed for adult monocytes treated with CORDHEAL (FIG. 9B). The adult cells displayed a similar diminished TNF-α expression in response to P. aeruginosa, but they were unable to mount a nitric oxide mediated response against the bacteria. This correlates well with what has been reported in the literature, where it is well documented that adult monocytes do not produce much nitric oxide in vitro, but that in vivo nitric oxide production is a significant microbial killing mechanism. Emerging evidence from both animal and human studies indicates that nitric oxide plays a key role in wound repair. The beneficial effects of nitric oxide on wound repair may be attributed to its functional influences on angiogenesis, inflammation, cell proliferation, matrix deposition, and remodeling. Consequently, the robust nitric oxide response exhibited by the monocytes pretreated with CORDHEAL in vitro to P. aeruginosa represents a novel mechanism of regulating nitric oxide production useful in methods of treatment of impaired wound healing.

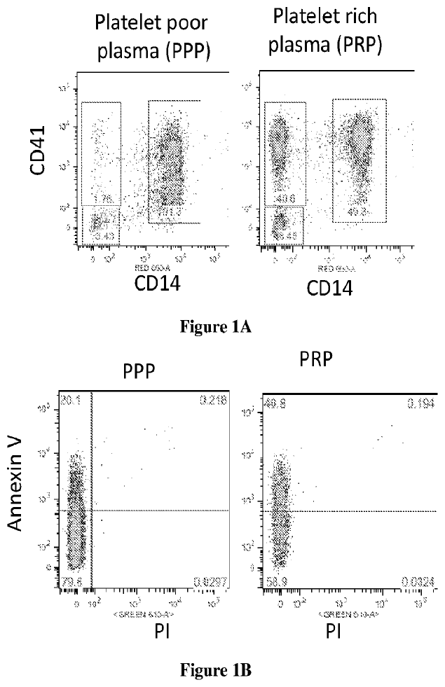

Monocyte purity of samples were an average of 87% pure (n=3) (FIG. 1A), and as a measure of the isolated monocyte viability, an average mortality of 0.25% apoptotic cells in fresh samples (n=3) was determined (FIG. 1B).

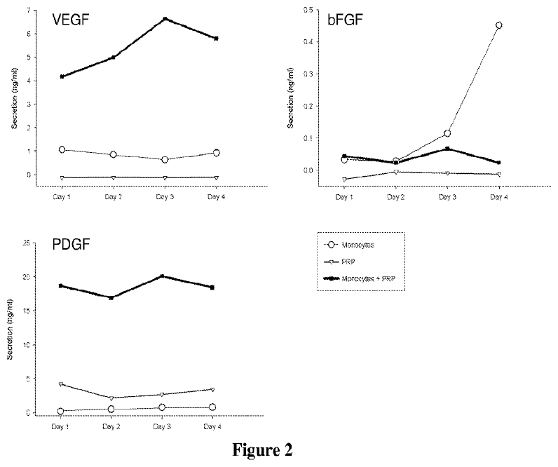

Chronic non-healing ulcers have a relatively low localized expression of growth factors, however umbilical platelet rich plasma (PRP) enhances monocyte growth factor production (FIG. 2).

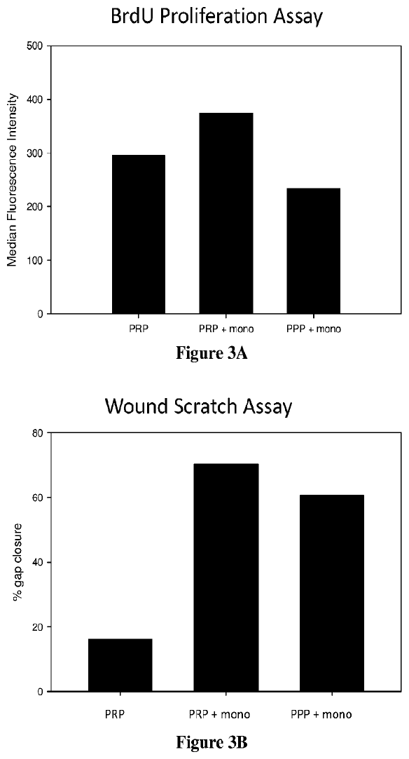

Umbilical platelet rich plasma (PRP) and monocytes show the highest effect in a BrdU proliferation assay and a wound scratch assay (FIGS. 3A and 3B).

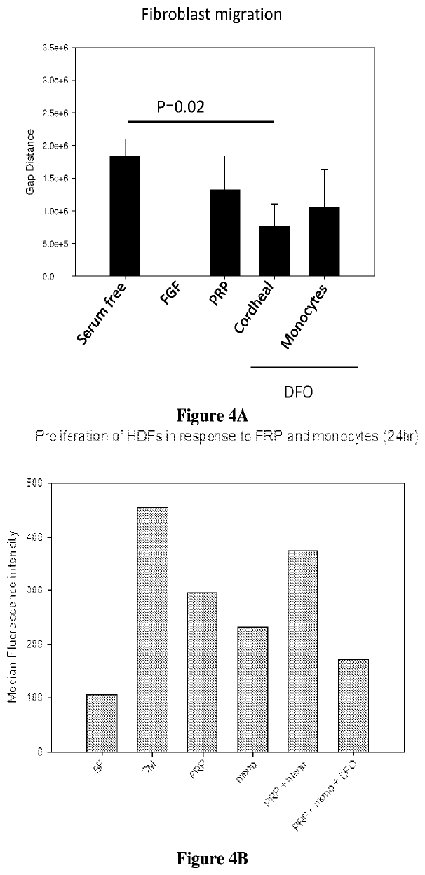

The results from a study investigating the effect of deferoxamine (DFO) stimulated monocytes on fibroblast function are shown in FIGS. 4A and 4B. CORDHEAL is a combination product comprising umbilical cord blood (UCB) derived monocytes that have been stimulated (e.g. monocytes stimulated with deferoxamine (DFO)) and platelet rich plasma (PRP). FIG. 4A shows the impact of DFO on fibroblast migration. As shown in FIG. 4B, various products were added to a fibroblast wound scratch assay, and the migration of fibroblasts into the scratch was measured after 24 hrs by microscopy. Treatment of monocytes with DFO enhanced fibroblast migration. FIG. 4B shows the impact of a CORDHEAL product comprising 500 μM DFO stimulated monocytes and PRP on human dermal fibroblasts (HDF) proliferation. In this study, the CORDHEAL product was added to fibroblast culture for 24 hrs and fibroblast proliferation was measured by BrdU incorporation. CORDHEAL modestly enhanced fibroblast proliferation. Modest proliferative effect is preferred, because hyperproliferation of fibroblasts is associated with formation of scar tissue.

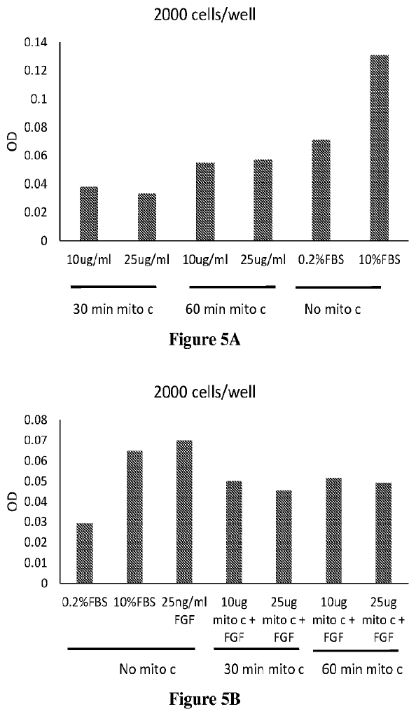

The results from two different studies investigating the effect of mitomycin c on human dermal fibroblast proliferation are shown in FIGS. 5A-5B. Fibroblast cells were plated out in complete media and then treated with mitomycin c (10 μg/ml or 25 μg/ml) for 30 or 60 min. Mitomcyin c was washed out and starvation media was added. Positive controls are 10% FBS and FGF (25 ng/ml). The ODs from the blank wells were subtracted out. Proliferation was measured by MTT assay at 17 hrs. Mitomycin c appears to inhibit fibroblast proliferation.

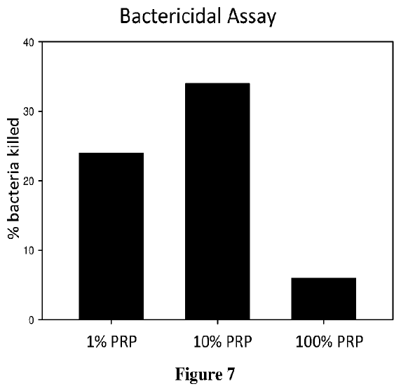

The results from a bactericidal assay investigating the ability of umbilical cord blood derived monocytes to kill Pseudomonas aeruginosa (P. aeruginosa) are shown in FIG. 7. Bactericidal activity of monocytes was assayed by measuring bacterial colony forming units of phagocytosed bacteria before and after bacterial killing. Monocytes exhibited optimal bactericidal activity against P. aeruginosa in the presence of 10% PRP compared to 1% or 100% PRP. This study was done in the absence of DFO.

The effect of DFO incubation on the TNF-α response to LPS and P. aeruginosa was studied. Monocytes and PRP from umbilical cord or adult blood was cultured in the presence or absence of DFO for 24 hrs then stimulated with LPS or P. aeruginosa (10⁶ CFU/ml-10⁸ CFU/ml) for 24 hrs. TNF-α was measured using ELISA. TNF-α was produced in large quantities in response to LPS and P. aeruginosa. DFO enhanced the TNF-α response to LPS, but dampened the TNF-α response to P. aeruginosa (FIGS. 8A and 8B). An extended incubation in DFO reduced donor to donor variability and yielded a differential TNF expression pattern in response to LPS and P. aeruginosa. The differential responses to LPS may be due to differences in MD2 in PRP.

The effect of DFO incubation on the nitric oxide (NO) response to LPS and P. aeruginosa was studied by measuring the breakdown product nitrite (NO₂—) in a Greiss reaction. Monocytes and PRP from umbilical cord or adult blood was cultured in the presence or absence of DFO for 24 hrs then stimulated with LPS or P. aeruginosa (10⁶ CFU/ml-10⁸ CFU/ml) for 24 hrs. NO was measured using Greiss assay. CBU derived monocytes significantly upregulated NO in response to P. aeruginosa compared to adult blood derived monocytes. DFO does not seem to impact NO responses (FIGS. 9A and 9 B, where *indicates P<0.05 compared to media. N=3 CBU donors, n=2 adult donors).

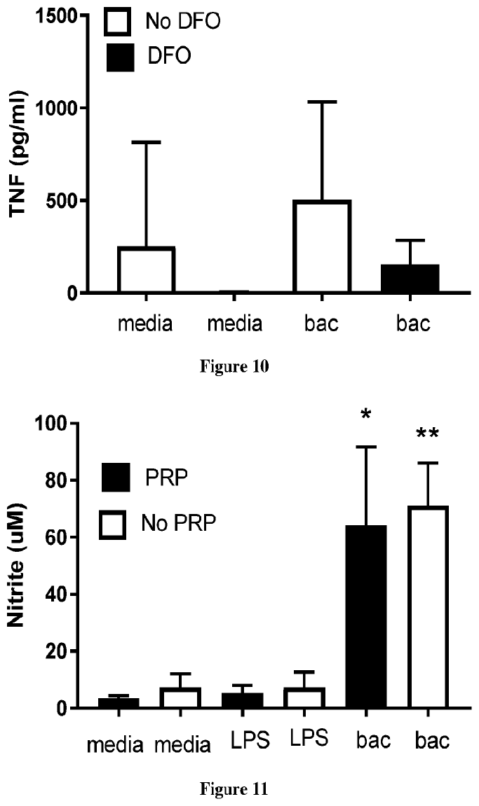

The mechanism of action of monocytes in vitro independent of PRP was studied. Monocytes were cultured in the presence or absence of DFO, then stimulated with LPS or P. aeruginosa (10⁶ CFU/ml) for 24 hrs. The results show that PRP is not required for both monocyte mediated TNF-α responses (FIG. 10), and monocyte mediated NO responses to LPS and P. aeruginosa (FIG. 11).

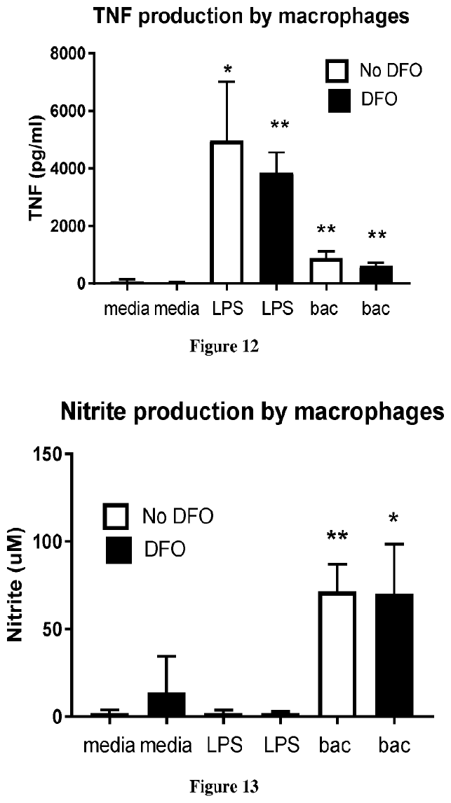

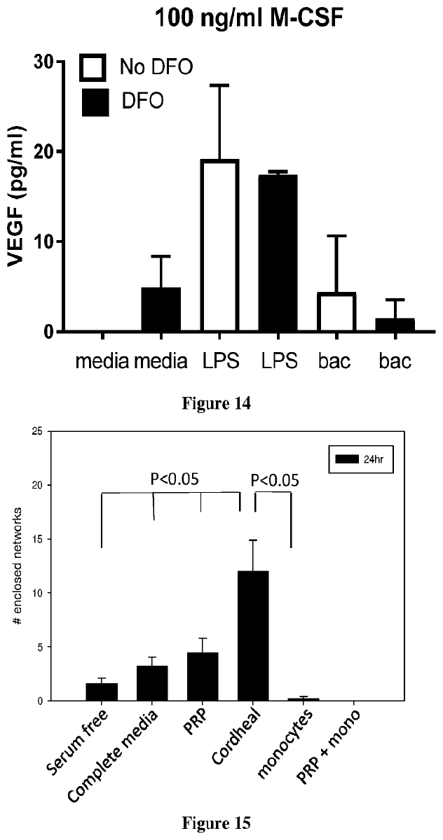

The potency of macrophages as compared to monocytes was investigated. Monocytes were differentiated into macrophages following DFO stimulation, and then stimulated with LPS or P. aeruginosa for 24 hrs. TNF-α was measured in supernatants. Monocytes were cultured in RPMI/10% AB serum DFO and MCSF (100 ng/ml) for the first 24 hrs. DFO was removed and the culture continued in the presence of MCSF (100 ng/ml) for 9 days. The media was replaced every 2-3 days. The results show that monocyte derived macrophages express similar levels of TNF-α (FIG. 12) and NO (FIG. 13) as compared to undifferentiated monocytes. However, monocyte derived macrophages express elevated levels of VEGF in response to LPS (FIG. 14).

The results from a MATRIGEL angiogenesis assay are shown in FIG. 15. The assay measured the ability of HUVECs to form endothelial tubules on a MATRIGEL matrix in response to stimuli. CORDHEAL (UCB derived monocytes stimulated with DFO and PRP) was added to HUVECs and the number of enclosed networks was counted after 24 hrs. The results showed that CORDHEAL synergistically enhances MATRIGEL endothelial tubule formation.

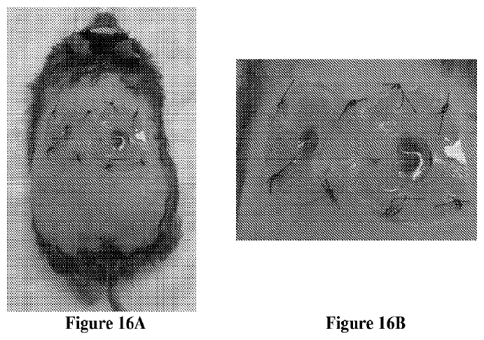

Images from an in vivo preclinical assay testing CORDHEAL (UCB derived monocytes stimulated with DFO and PRP) in a murine splinted excisional biopsy model are shown in FIGS. 16A and 16B. A splinted excisional biopsy wound was created by 2×5 mm punch biopsies on the back of genetically diabetic mice. Diabetic mice have elevated and uncontrolled blood glucose, are obese, and exhibit slower wound healing compared to wild type. A silicone splint was glued and sutured on to the wound to prevent contraction. The silicone splint slows the rate of wound contraction, allowing wound closure to occur by re-epithelialization and granulation tissue formation, which more accurately resembles human wound healing. CORDHEAL or a control material was applied twice per week. Data revealed that the overall health of the CORDHEAL treated group was better as compared to sham controls as measured by body weight.

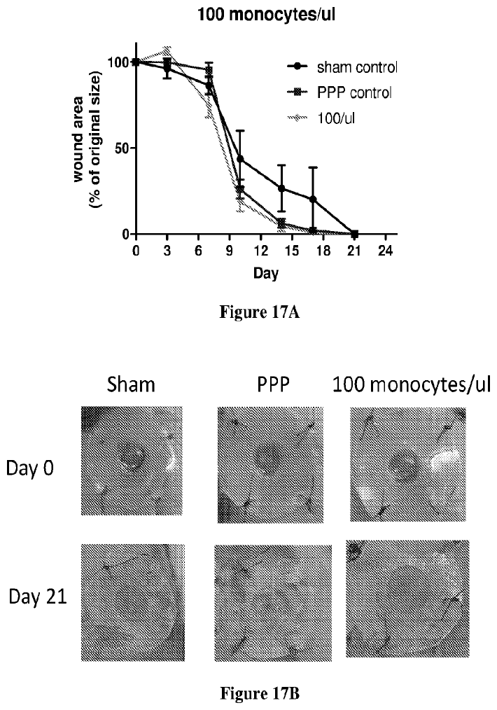

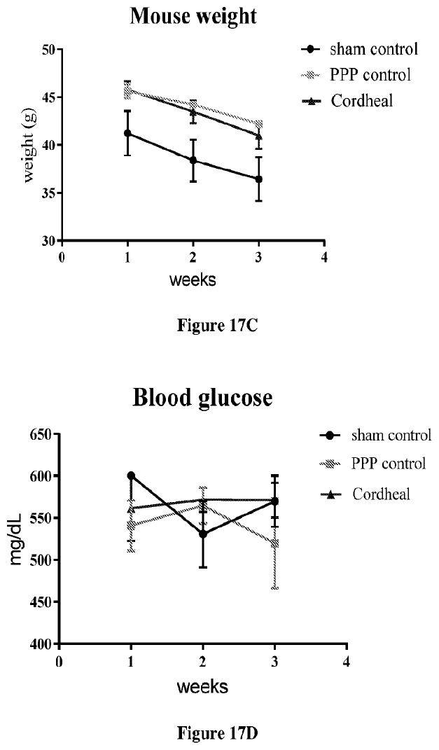

FIGS. 17A-17D show data where genetically diabetic (db/db) mice received two full thickness wounds on the dorsum which were splinted open to promote secondary intention. CORDHEAL was applied at the time of wounding, and twice a week afterwards. There were no adverse reactions to CORDHEAL, and the treatment group showed modest improvement in healing at the early time points. Weight and fasting blood glucose were monitored weekly. N=3 mice per group.

Overall, CORDHEAL displayed in vitro and in vivo functionality associated with the ability to enhance wound healing, and an enhancement of microbial killing as compared to adult monocytes.

1. A method for producing a pharmaceutically acceptable wound healing composition for administration to a patient in need thereof comprising:
producing platelet rich plasma from umbilical cord blood;
isolating monocytes from umbilical cord blood;
stimulating the isolated monocytes with deferoxamine in culture in vitro; and
combining the platelet rich plasma and the stimulated monocytes into a pharmaceutically acceptable wound healing composition.

2. The method of claim 1, wherein the pharmaceutically acceptable composition forms a gel.

3. The method of claim 1, further comprising before the combining step:
cryopreserving the platelet rich plasma and the isolated monocytes;
thawing the platelet rich plasma and the monocytes; and then combining the platelet rich plasma and the monocytes.

4. The method of claim 1, wherein the combining step occurs before the stimulating step.

5. The method of claim 1, wherein mesenchymal stromal cells are added to the healing composition.

6. The method of claim 1, further comprising after the stimulating step, adding thrombin to the monocytes.

7. The method of claim 1, further comprising before the stimulating step, enriching the monocytes with DMEM then RPMI/human AB serum and M-CSF.

8. A therapeutic composition for wound healing comprising:
platelet rich plasma derived from umbilical cord blood; and
monocytes derived from umbilical cord blood,
wherein the monocytes have been stimulated with deferoxamine in culture in vitro.

9. The composition of claim 8, further comprising mesenchymal stromal cells.

10. A method for treating a non-healing wound in a subject, comprising administering to the subject a composition comprising a therapeutically effective amount of umbilical cord blood derived platelet rich plasma and umbilical cord blood derived monocytes previously stimulated with deferoxamine in culture in vitro.

11. The method of claim 10, wherein the non-healing wound is a chronic wound.

12. The method of claim 10, wherein the non-healing wound is a diabetic ulcer, a decubitus ulcer, a venous ulcer, an arterial ulcer, an infectious ulcer, a burn ulcer, a trauma-induced ulcer, or a surgical wound.

13. The method of claim 10, wherein the platelet rich plasma and the monocytes are derived from allogeneic umbilical cord blood.

14. The method of claim 10, wherein the therapeutically effective amount of umbilical cord blood derived platelet rich plasma and umbilical cord blood derived monocytes stimulated with deferoxamine further comprises mesenchymal stromal cells.
